(19) United States
(12) Patent Application Publication
(10) Pub. No.: US 2022/0133610 A1
(43) Pub. Date: May 5, 2022

(54) SUNSCREEN COMPOSITIONS

(71) Applicant: BEIERSDORF AG, Hamburg (DE)

(72) Inventors: Yongyi ZHANG, New Providence, NJ (US); Sherilee Jane BACKMAN, Morganville, NJ (US)

(21) Appl. No.: 17/310,238

(22) PCT Filed: Jan. 30, 2020
(86) PCT No.: PCT/US2020/015834

§ 371 Date: Jul. 27, 2021

Related U.S. Application Data
(60) Provisional application No. 62/800,236, filed on Feb. 1, 2019.

(51) Int. Cl. A61K 8/35; A61K 8/02; A61K 8/26; A61K 8/25; A61K 8/19; A61K 8/06; A61K 8/81; A61K 8/04; A61Q 17/04

(52) U.S. Cl. CPC A61K 8/35; A61K 8/0266; A61K 8/26; A61K 8/25; A61K 2800/436; A61K 8/062; A61K 8/8147; A61K 8/042; A61Q 17/04; A61K 8/19

(57) ABSTRACT

Disclosed are compositions which are suitable for use as sunscreen lotions and creams. The compositions have organic filters and special effects pigments, providing both sun protection and aesthetically pleasing visual effects to the skin. The compositions may also be formulated to form high oil load emulsions that exhibit both a quick-break effect, as well as physical stability at different temperature levels. Methods of manufacturing and using such compositions are also disclosed.

### FIELD

The present disclosure relates generally to sun protection compositions, and more specifically to compositions that include special effect pigments to provide both sun protection and aesthetically pleasing visual effects to the skin.

### BACKGROUND

Special effect pigments are generally used in personal care products and other applications to achieve a soft shine, a satin luster, or a brilliant sparkling or metallic appearance.

Pearlescent pigments comprise a variety of formats, and can include monocrystalline pearl luster pigments such as bismuth oxychloride, mica platelets, and layer-substrate pigments in which a coating is distributed over and bound to the surface of a substrate, such as when a lamellar layer of titanium dioxide coats a mica platelet.

Such layer-substrate pigments often contain a substrate that may or may not confer pearlescence itself. For example, mica and synthetic fluorphlogopite (synthetic mica) platelets are often inherently pearlescent. Borosilicate beads often are not.

Examples of substrates can include platelets or flakes of mica, synthetic fluorphlogopite, metal silicates and glass, silica, and metal powders including alumina, aluminum, silver and copper powders.

Metal powders can also be used as neat particles to create special metallic effects.

Layer-substrate pigments often contain metal oxides, such as titanium oxide and iron (III) oxides. However, regulations in the United States place many restrictions on types of pigments and other materials for skin use.

For example, titanium dioxide is very often used as a surface treatment on color substrates to refract and reflect specific wavelengths of light based on the thickness of its deposition, to enable the human eye to perceive the specific colors associated with those wavelengths. However, in the US, titanium dioxide cannot be used in combination with avobenzone, so the playing field of available pigments was greatly reduced by the elimination of this colorant option.

Silver powder generally imparts a silvery shimmer effect. However, silver is an example of an ingredient not permitted in the United States for over-the-counter topical products. Thus, silver-based products would not be desirable for skin use products in the United States.

Certain other ingredients raise consumer controversy and generally need to also be avoided. Thus, what is needed in the art are sunscreen formulations that use organic filters and pearlescent pigments that provide both sun protection and aesthetically pleasing visual effects to the skin.

### BRIEF SUMMARY

Provided herein are compositions, suitable for use as sunscreen lotions and creams, that use organic filters and pearlescent pigments, providing both sun protection and aesthetically pleasing visual effects to the skin.

In some aspects, provided is a composition, comprising: avobenzone; and a plurality of special effects pigments. In some embodiments, at least two of the special effect pigments are (i) a first pigment comprising a layered or deposited metal oxide or salt, or other material, on a substrate, and (ii) a second pigment comprising a mineral colorant platelet. In some variations, the composition is free of titanium dioxide. In certain variations, the metal oxide is iron oxide. In one variation, the metal salt comprises bismuth oxychloride. In another variation, the other material comprises silica. In another variation, the substrate comprises mica or synthetic fluorphlogopite or silica, or a combination thereof.

In some embodiments that may be combined with any of the foregoing, the composition further comprises a plurality of emulsifiers. In some embodiments, one of the emulsifiers comprises polyacrylate. In some embodiments, another of the emulsifiers comprises at least one glucoside and at least one alcohol. In certain embodiments of the foregoing, the composition is an oil-in-water emulsion. In some variations, the composition is an oil-in-water quick break emulsion system.

In other aspects, provided is a method of protecting skin, scalp, and/or hair of a human from the harmful radiation emitted by the sun.

In yet other aspects, provided is an article of manufacture comprising: a container comprising any of the compositions described herein; and a label containing instructions for use of the composition.

### DESCRIPTION OF THE FIGURE

The present application can be understood by reference to the following description taken in conjunction with the accompanying FIGURE.

FIG. 1 is a 1-dimensional perceptual map comparing performance during manipulation (Firmness, Stickiness, Peaking, Integrity of Shape & Thickness) with performance during rubout (Wetness, Spreadability, Slipperiness, Oily, Absorbency).

### DETAILED DESCRIPTION

The following description sets forth exemplary methods, parameters and the like. It should be recognized, however, that such description is not intended as a limitation on the scope of the present disclosure but is instead provided as a description of exemplary embodiments.

Provided herein are compositions with formulation aesthetics that target beauty sector consumers as well as sunscreen sector consumers. In some embodiments, the compositions, when applied on skin, have attractive shimmer and glow effects that provide a product mass tone that disappears on the skin. For example, in one variation, the composition imparts a golden, rosy, coppery, bronzed, or rose gold, shimmering effect on the skin. The composition can be applied to all skin types without altering skin tone or creating ashiness or chalkiness. Further, in certain variations, the compositions can provide a cooling, light feel that resembles the attributes of a moisturizer more than those of conventional beachwear.

In some aspects, the compositions provided are made up of one or more organic active agents and one or more special effect pigments. In certain aspects, the organic active agents are or comprise organic sunscreen active agents, and the composition is a sunscreen composition. In some embodiments of the foregoing, at least one of the organic actives is avobenzone. In other embodiments of the foregoing, the composition includes at least two special effect pigments. At least one of the special effect pigments comprises a layered-substrate pigment. Other special effect pigments may comprise an untreated platelet colorant.

The various ingredients of the compositions, as well as properties, methods of manufacturing and using the compositions are further described below.

Active Agents

The active agents are typically skincare active agents. In some variations, skincare active agents may include materials regarded as acceptable for use as active skin-protecting ingredients. A skincare active agent may include, for example, skin protectant and/or anti-aging agent. Approval by a regulatory agency may sometimes be required for inclusion of active agents in formulations intended for human contact, including for example, sunscreen active ingredients or skin protectant ingredients.

The active agents present in the composition may differ based on the purpose of the composition. For example, a sunscreen composition comprises sunscreen active agents. In some variations, the active agents are or comprise organic active agents.

Any suitable sunscreen active agent may be part of the sunscreen composition. Approved sunscreen active agents in the United States and elsewhere include, for example, para-aminobenzoic acid, avobenzone, cinoxate, dioxybenzone, homosalate, menthyl anthranilate, octocrylene, octyl methoxycinnamate, octyl salicylate, oxybenzone, 2-ethylhexyl 4-(dimethylamino)benzoate (e.g., Padimate 0), phenylbenzimidazole sulfonic acid, octisalate, sulisobenzone, trolamine salicylate, titanium dioxide and zinc oxide. Several other sunscreen active ingredients are accepted for use in other countries. Examples from outside the U.S. include Tinosorb M, Tinosorb S, Uvinul T-150, UVA sorb HEB, Uvinul A Plus, Neo Heliopan AP, and Neo Heliopan MBC.

As described above, the compositions provided herein comprise at least avobenzone. In some embodiments, the compositions further comprise additional active agents. In some variations, the compositions further comprise additional organic active agents. In certain variations, the compositions comprise at least one additional organic active agent selected from octocrylene, octisalate, and homosalate, or any combination thereof. In one variation, the compositions comprise avobenzone, octocrylene, octisalate, and homosalate.

In certain embodiments, in addition to the skin-active ingredients already described, the compositions provided comprise one or more additional skin-active ingredients, such as a humectant and moisturizing ingredients, an anti-aging agent, a depigmenting agent, an anti-wrinkle agent, or an agent that treats oily skin. Additional active agents may include, for example, adenosine, hyaluronic acid, lanolin, citric acid, malic acid, lactic acid, tartaric acid, salicylic acid, a vitamin, a retinoid, retinol, retinoic acid, a carotenoid, an amino acid, a protein, an enzyme, and a coenzyme.

It is typical to use combinations of two or more skincare active agents in a formulation. The amount of skincare active agent or agents may be present in an amount that is consistent with the guidelines of the FDA or other regulatory bodies. The use of a combination of active agents is especially true for sunscreen formulations to achieve higher levels of ultraviolet absorption or to provide useful absorption over a wider range of ultraviolet wavelengths than can be the case with a single active component. Preferably, the sunscreen active agent or agents is present in an amount that is consistent with the FDA sunscreen monograph for sunscreen active agent or agents that are believed to provide the requisite SPF in accordance with the FDA monograph for such sunscreens. Other skin care active agents may include, for example, sunless tanning active agents, skin protectant active agent emollients, and insect repelling agents.

In some variations, the one or more active agents are present in the compositions herein in amounts of from 10% to 40%, from 20% to 40%, or from 20% to 35% by weight of the composition.

### Special Effect Pigments

The compositions provided herein further comprise at least one special effect pigment. In some embodiments, the compositions comprise at least two special effect pigments. In some embodiments, the compositions comprise at least three or more special effect pigments.

Layered-Substrate Pigment

In some variations, the special effect pigment comprises at least one colorant on at least one substrate. Suitable substrates may include, for example, glass, silica, silicate, alumina, natural or synthetic mica or synthetic fluorphlogopite, or any combination thereof. In one variation, the substrate comprises natural mined mica. In another variation, the substrate comprises synthetic fluorphlogopite. In another variation, the substrate comprises silica. In yet another variation, the substrate comprises mica and synthetic fluorphlogopite. In yet another variation, the substrate comprises mica or synthetic fluorphlogopite and silica.

In some variations, one of the special effect pigments comprises at least one colorant layered or deposited on at least one substrate. Suitable colorants include, for example, metal oxides, metal salts, organic pigments, and other materials. In one embodiment, the colorant comprises a metal oxide. In some variations, the metal oxide is or comprises iron oxide. In another embodiment, the colorant comprises a metal salt. In some variations, the metal salt is bismuth oxychloride. In another embodiment, the colorant comprises other materials such as silica, ultramarines and flake pigments. In some variations, the other material is or comprises silica. In some variations of the foregoing, such metal oxide pigment does not contain titanium dioxide.

Pearlescent Platelet Pigment

In other embodiments, another of the special effect pigments comprises a pearlescent platelet. In certain embodiments, the pearlescent platelet comprises a pearlescent pigment. In some variations, the pearlescent pigment comprises bismuth oxychloride. In other variations, the pearlescent pigment comprises mica. In other variations, the pearlescent pigment comprises synthetic fluorphlogopite.

The substrate may be in any suitable form known in the art. For example, in certain variations, the substrate is in the form of flakes, spheres, or platelets. In one variation, the substrate is in the form of platelets. In another variation, the substrate is in the form of flakes. In some variations of the foregoing, at least one colorant is deposited on the platelets and/or flakes.

In other variations of the foregoing, the special effect pigment has a particle size of from 5 μm to 250 μm; or from 5 μm to 200 μm; or from 5 μm to 20 μm.

In yet other variations of the foregoing, the special effect pigments ranging in particle size from about 1 μm to about 25 μm may impart a satiny visual appearance. In other variations of the foregoing, the special effect pigments ranging in particle size from about 10 μm to about 60 μm may impart a lustrous visual appearance. In other variations of the foregoing, the special effect pigments ranging in particle size from about 20 μm to about 250 μm may impart a glittery visual appearance.

In some variations of the foregoing, the total pigment load in the composition ranges from less than about 1% by weight to about 25% by weight, or from less than about 1% by weight to about 5% by weight, or from about 5% by weight to about 25% by weight.

### Emulsion System

In some embodiments, the compositions are oil-in-water (O/W) emulsions with quick break properties. In certain embodiments, the compositions are quick-break, high oil load emulsions.

In some variations, the compositions have an oil load of at least 5%, at least 10%, at least 15%, at least 20%, at least 25%, or at least 30%; or between 5% and 50%, between 10% and 50%, between 15% and 50%, between 20% and 50%, between 25% and 50%, between 30% and 50%, between 35% and 50%, between 40% and 50%, between 5% and 45%, between 10% and 45%, between 15% and 45%, between 20% and 45%, between 25% and 45%, between 30% and 45%, between 5% and 40%, between 10% and 40%, between 15% and 40%, between 20% and 40%, between 25% and 40%, between 30% and 40%, based on the total weight of the composition.

Various emulsifiers are used in the compositions.

Polyacrylate Emulsifiers

In one embodiment, one of the emulsifiers comprises polyacrylate.

In some variations, the polyacrylate emulsifier comprises a crosspolymer of a sulfonic acid acrylamide monomer or a salt thereof, and an alkyl methacrylate monomer or a salt thereof. In certain variations, the polyacrylate emulsifier comprises a crosspolymer of an ammonium salt of a sulfonic acid acrylamide monomer, and an alkyl methacrylate monomer.

In other variations, the polyacrylate emulsifier comprises a crosspolymer of: a monomer of formula (M1) and a monomer of formula (M2), wherein:

the monomer of formula (M1) is:

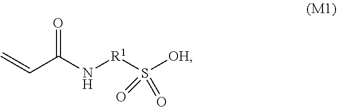

or a salt thereof, wherein R¹ is -alkyl-;

the monomer of formula (M2) is:

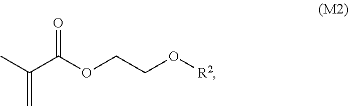

or a salt thereof, wherein R² is alkyl.

In certain variations of the foregoing, the monomer of formula (M1) is an ammonium salt. In certain variations of the foregoing, R¹ is a branched alkylene. In one variation, R¹ is —C₁₋₁₀ alkylene. In certain variations, R² is C₁₅₋₃₀ alkyl.

Suitable polyacrylate emulsifiers may include, for example, ammonium acryloyldimethyltaurate/beheneth-25 methacrylate crosspolymer (e.g., Aristoflex HMB); acrylates/palmeth-25 acrylate copolymer (e.g., Rheostyl E-Light); sodium acrylate/acryloyldimethyltaurate/dimethylacrylamide crosspolymer and isohexadecane and polysorbate 60 (e.g., Simulgel SMS 88); ammonium acryloyldimethyltaurate/carboxyethyl acrylate crosspolymer (e.g., Aristoflex TAC); acrylate/C10-30 alkyl acrylate crosspolymer (e.g., Pemulen TR-1 and TR-2, Protamer Z-21); acrylates copolymer (e.g., Emul 33); carbomer (e.g. Ashland 981 Carbomer); and acrylic acid/vinyl pyrrolidone crosspolymer (e.g., UltraThix P-100).

A combination of the foregoing polyacrylate emulsifiers may also be used in the compositions described herein.

Glucoside/Alcohol Emulsifiers

In another embodiment, another of the emulsifiers comprises at least one glucoside and at least one alcohol. In one embodiment, the alcohol is a fatty alcohol. In certain variations, the alcohol is a C₈₊ fatty alcohol. In another variation, the alcohol is a C₁₂ to C₂₅ fatty alcohol. In some variations, the length of the alcohol and the glucoside is the same or similar.

In some variations, the glucoside/alcohol emulsifier comprises at least one glucoside and at least one fatty alcohol. In certain variations, the glucoside/alcohol emulsifier comprises one, two or three glucosides. In another variation that may be combined with the foregoing, the glucoside/alcohol emulsifier comprises one, two, or three fatty alcohols.

In other variations, at least one glucoside is a glucoside of formula (G1):

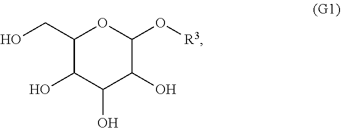

or a salt thereof, wherein R³ is alkyl.

In one variation, the glucoside of formula (G1) is a glucoside of formula (G1a):

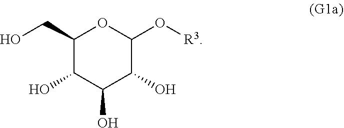

In certain variations of the foregoing, R³ is a C₈₊ alkyl. In another variation, R³ is a C₁₂ to C₂₅ alkyl.

In some embodiments, the glucoside/alcohol emulsifiers may include additional components, such as seed oil and stearates.

Suitable glucoside/alcohol emulsifiers may include, for example, arachidyl alcohol and behenyl alcohol and arachidyl glucoside (e.g., Montanov 202); cetearyl glucoside and cetearyl alcohol (e.g., Montanov 68, SpecSufc SC-M68); coco-glucoside and coconut alcohol (e.g. Montanov S); myristyl alcohol and myristyl glucoside (e.g., Montanov 14); cetearyl alcohol and coco-glucoside (e.g., Montanov 82); C14-22 alcohols and C12-20 alkyl glucoside (e.g., Montanov L); cetearyl glucoside and sorbitan olivate and cetearyl alcohol (e.g., Beautyderm K10); cetearyl alcohol and polysorbate 60 and cetearyl glucoside (e.g., Uniox Cristal); hydroxyethyl acrylate/sodium acryloyldimethyl taurate copolymer and cetearyl alcohol and arachidyl alcohol and coco-glucoside and behenyl alcohol and arachidyl glucoside (e.g., Simplynov 365); cetearyl alcohol and glyceryl stearate and sorbitan stearate and cetearyl glucoside (e.g., Emulpharma Coreosome); and Cannabis sativa seed oil or other oil and C12-18 alkyl glucoside and glyceryl stearate and cetearyl alcohol and stearic acid (e.g., AE EmulsiPure Hempseed).

It is understood that any description of the polyacrylate emulsifiers may be combined with any descriptions of the glycoside/alcohol emulsifiers the same as if each and every combination were individually listed. For example, in one variation, at least two of the emulsifiers in the compositions are: (i) ammonium acryloyldimethyltaurate/beheneth-25 methacrylate crosspolymer, and (ii) arachidyl alcohol, behenyl alcohol and arachidyl glucoside.

### Other Ingredients

In some embodiments, the compositions herein include additional ingredients, such as solvents, film formers, SPF boosters, emulsification and stabilization system components, skin feel enhancers, excipients, vitamins, and preservation system components.

Solvents

In certain embodiments, one or more solvents are present in the compositions described herein. In some variations, the solvent comprises water. In one embodiment, from 40% to 70%, based on the total weight of the composition, of water is present. In another embodiment, from 40% to 70%, based on the total weight of the composition, of water and alcohol are present.

In some variations, little to no alcohol is present in the compositions. In certain variations, less than 15%, less than 10%, less than 5%, less than 1%, based on the total weight of the composition, of alcohol is present. In one variation, the composition has no alcohol. In variations of the composition where alcohol is present, suitable alcohols, such as ethanol, may be used.

Film Formers

In certain embodiments, one or more film formers are present in the compositions described herein. In some variations, a combination of film formers is used.

In some embodiments, the one or more film formers are present in an amount of not more than about 7.5%, about 5%, about 4%, about 3%, about 2%, about 1%, about 0.5%, about 0.25%, or about 0.1% by weight. In certain embodiments, the one or more film formers are present in an amount of about 3 wt % based on the total weight of the sunscreen formulation.

In some variations, the one or more film formers are present in a dry matter content ranging from 0.1% to 60%, from 0.5% to 40%, or from 1% to 30% by weight relative to the total weight of the composition.

In one embodiment, the film formers are a polymer capable of forming, on its own or in the presence of a film-forming aid, a continuous and adherent film on a support, for instance on the keratinous materials.

In some variations, the film formers used are capable of forming a hydrophobic film. In certain variations, the film formers are polymers capable of forming a hydrophobic film having a solubility in water at 25° C. of less than 1% by weight.

In other variations, the film formers may be synthetic polymers of the free-radical type or of the polycondensate type, polymers of natural origin and mixtures thereof. Free-radical film-forming polymers include polymers obtained by polymerization of monomers with for instance, ethylenic unsaturation, each monomer being capable of homopolymerizing (in contrast to polycondensates).

In other variations, the film formers may be of the free-radical type, for example, vinyl polymers or copolymers. Examples include acrylic polymers. In one variation, the vinyl film-forming polymers may result from the polymerization of ethylenically unsaturated monomers having at least one acid group and/or esters of these acid monomers and/or amides of these acid monomers. In another variation, as a monomer carrying an acid group, there may be used C.B-ethylenic unsaturated carboxylic acids such as acrylic acid, methacrylic acid, crotonic acid, maleic acid and itaconic acid. For example, (meth)acrylic acid and itaconic acid may be used. In yet another variation, the esters of acid monomers may be chosen from the esters of (meth)acrylic acid (also called (meth)acrylates), for example alkyl, such as C—Co, for instance C—C, alkyl, (meth)acrylates, aryl, such as Co—Co aryl, (meth)acrylates, hydroxyalkyl, for instance C—C hydroxyalkyl, (meth)acrylates. Examples of alkyl (meth)acrylates may include methyl methacrylate, ethyl methacrylate, butyl methacrylate, isobutyl methacrylate, 2-ethyl hexyl methacrylate, lauryl methacrylate and cyclohexyl methacrylate. Examples of hydroxyalkyl (meth)acrylates may include hydroxyethyl acrylate, 2-hydroxypropyl acrylate, hydroxyethyl methacrylate and 2-hydroxypropyl methacrylate. Examples of aryl (meth)acrylates may include benzyl acrylate and phenyl acrylate. In certain variations the alkyl group of the esters may be either fluorinated or perfluorinated (e.g., some or all of the hydrogen atoms of the alkyl group may be substituted with fluorine atoms). Examples of amides of the acid monomers may include (meth)acrylamides, such as N-alkyl(meth)acrylamides, and for instance, of a C—C alkyl. Examples of N-alkyl(meth)acrylamides may include N-ethylacrylamide, N-t-butylacrylamide, N-t-octylacrylamide and N-undecylacrylamide.

In some variations, the vinyl film formers may also result from the homopolymerization or copolymerization of monomers chosen from vinyl esters and styrene monomers. For example, these monomers may be polymerized with acid monomers and/or their esters and/or their amides, such as those mentioned above. Examples of vinyl esters may include vinyl acetate, vinyl neodecanoate, vinyl pivalate, vinyl benzoate and vinyl t-butylbenzoate. Examples of styrene monomers may include styrene and alpha-methylstyrene. In other variations, any acrylic and vinyl monomers (including the monomers modified by a silicone chain) may be used.

In other variations, the film formers are film-forming polycondensates, such as polyurethanes, polyesters, polyester amides, polyamides, epoxy ester resins, and polyureas. In one variation, the polyamides include, for example, polyamide-8. In another variation, the polyurethanes may be chosen from anionic, cationic, nonionic and/or amphoteric polyurethanes, polyurethane-acrylics, polyurethane-polyvinylpyrrolidones, polyester-polyurethanes, polyether-polyurethanes, polyureas, and polyurea-polyurethanes. In certain variations, the polyesters may be obtained by polycondensation of dicarboxylic acids with polyols, such as diols. In yet other variations, the dicarboxylic acids may be aliphatic, alicyclic and/or aromatic. Suitable acids may include, for example, oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornanedicarboxylic acid, diglycolic acid, thiodipropionic acid, 2,5-naphthalenedicarboxylic acid and 2,6-naphthalenedicarboxylic acid. These dicarboxylic acid monomers described herein may be used alone or in combination with at least two dicarboxylic acid monomers. Examples of monomers that may be used are phthalic acid, isophthalic acid and terephthalic acid. In other variations, the diols may be chosen from aliphatic, alicyclic and/or aromatic diols. For example, the diols may be chosen from: ethylene glycol, diethylene glycol, triethylene glycol, 1,3-propanediol, cyclohexanedimethanol and 4-butanediol. Examples of polyols may include glycerol, pentaerythritol, sorbitol, and trimethylolpropane.

In some variations, the polyester amides may be obtained in a manner similar to the polyesters, by polycondensation of diacids with diamines or amino alcohols. As diamines, there may be used ethylenediamine, hexamethylenediamine, meta- and/or para-phenylenediamine. As aminoalcohols, monoethanolamine may be used. In certain variations, the polyester may, in addition, comprise at least one monomer carrying at least one —SOM group, wherein M is chosen from hydrogen atoms, ammonium ions and metal ions (such as for example Na, Li, K, Mg, Ca, Cu, Fe ions). In other variations, the at least one monomer may be a bifunctional aromatic monomer comprising such an —SOM group. In certain variations, the aromatic ring of the bifunctional aromatic monomer carrying an —SOM group as described above may be chosen, for example, from benzene, naphthalene, anthracene, diphenyl, oxydiphenyl, sulphonyl diphenyl and methylenediphenyl rings, sulphoisophthalic acid, sulphoterephthalic acid, sulphophthalic acid, and 4-sulphonaphthalene-2,7-dicarboxylic acid.

In other variations, copolymers may be used. In certain variations, the copolymers are based on isophthalate or sulphoisophthalate. In certain variations, the copolymers may be obtained by condensation of diethylene glycol, cyclohexanedimethanol, isophthalic acid and sulphoisophthalic acid. In certain variations, the optionally modified polymers of natural origin may be chosen from shellac resin, sandarac gum, dammars, elemis, copals, cellulosic polymers and mixtures thereof.

In some embodiments, the film formers may be present in the form of particles in aqueous dispersion, generally known as latex or pseudolatex. The techniques for preparing these dispersions are well known to persons skilled in the art.

In other variations, the film formers are an aqueous dispersion of film-forming polymers. In certain variations, the film formers are dispersions of polymers resulting from the free-radical polymerization of one or more free radical monomers inside and/or partly at the surface, of preexisting particles of at least one polymer chosen from polyurethanes, polyureas, polyesters, polyesteramides and/or alkyls. These polymers are generally called hybrid polymers.

In other variations, the film formers may be a water-soluble polymer and is present in the aqueous phase of the composition in solubilized form. Examples of film-forming water-soluble polymers may include proteins. In one variation, the proteins are of plant origin (such as wheat or soya bean proteins), proteins of animal origin (such as keratins, for example keratin hydrolysates and sulphonic keratins), as well as anionic, cationic, amphoteric or nonionic polymers of chitin or chitosan. In other variations, the polymers are cellulose polymers such as hydroxyethyl cellulose, hydroxypropyl cellulose, methyl cellulose, ethyl hydroxyethyl cellulose, carboxymethyl cellulose, and quaternized derivatives of cellulose. In yet other variations, the polymers are acrylic polymers or copolymers such as poly acrylates or polymethacrylates. In yet other variations, the polymers are vinyl polymers, such as polyvinylpyrrolidones, copolymers of methyl vinyl ether and maleic anhydride, copolymers of vinyl acetate and crotonic acid, copolymers of vinylpyrrolidone and vinyl acetate; copolymers of vinylpyrrolidone and caprolactam; polyvinyl alcohol: In other variations, the polymers are optionally modified polymers of natural origin, such as gum arabic, guar gum, xanthan derivatives, karaya gum, glycoaminoglycans, hyaluronic acid and its derivatives; shellac resin, sandarac gum, dammars, elemis, copals; alginates and carrageenans; deoxyribonucleic acid; and muccopolysaccharides such as chondroitin sulphates.

In one embodiment, the film formers are selected from vinyl polymers, vinyl copolymers (such as acrylic polymers or acrylates), fluorinated or perfluorinated acrylates, acrylamides, vinyl esters, styrene esters, silicone-modified vinyl polymers, vinyl copolymers, acrylates, acrylamides, other vinyl esters, styrene esters, polyurethanes, polyesters, polyester amides, polyamides, epoxy ester resins, polyureas, optionally modified polymers of natural origin, shellac resin, sandarac gum, dammars, elemis, copals, cellulosic polymers, hydrolyzed starches, latexes, abietates, hybrid polymers, proteins, anionic, cationic, amphoteric or nonionic polymers of chitin or chitosan, cellulose polymers, and pullulan.

In other variations, the film formers are selected from anionic, cationic, nonionic and/or amphoteric polyurethanes, polyurethane-acrylics, polyurethane-polyvinylpyrrolidones, polyester-polyurethanes, polyether-polyurethanes, polyureas, and polyurea-polyurethanes. In one variation, the film formers are diols.

In another variation, the film formers are selected from wheat or Soya bean proteins; proteins of animal origin such as keratins, for example keratin hydrolysates and sulphonic keratins.

In yet another variation, the film formers are selected from hydroxyethyl cellulose, hydroxypropyl cellulose, methyl cellulose, ethyl hydroxyethyl cellulose, carboxymethyl cellulose, and quaternized derivatives of cellulose.

In yet another variation, the film formers are selected from bis-octyldodecyl dimer dilinoleate/propanediol copolymer and octyldodecyl/glyceryl hydroxy stearate dilinoleate dimethicone copolymer.

SPF Boosters

In certain embodiments, one or more SPF boosters are present in the compositions described herein. In some variations, a combination of SPF boosters is used.

In some embodiments, the one or more SPF boosters are present in an amount of from 0.1% to 10%, or from 0.1% to 8%; or about 10%, about 9%, about 8%, about 7%, about 6%, about 5%, about 4%, about 3%, about 2%, about 1%, about 0.5%, about 0.2%, or about 0.1% by weight of the total composition.

Suitable SPF boosters may include butyloctyl salicylate, ethylhexyl methoxycrylene, styrene/acrylates copolymer, styrene/acrylates copolymer (and) acrylates copolymer, glycogen, acrylates/methacryloyloxyethyl phosphate copolymer, dimethyl capramide, neopentyl glycol diethylhexanoate (and) neopentyl glycol diisostearate, Daucus carota sative (carrot) root extract (and) Helianthus annuus (sunflower) seed oil, Argania spinosa kernel oil (and) tocopheryl acetate (and) bisabolol, PVP (and) VP/Eicosene copolymer, VP/eicosene copolymer, hydrophobically modified starches, silica beads, PMMA beads, borosilicate beads, polyurethane beads, diatomaceous earth, betonite and hectorite clays and any combination thereof.

Emulsifiers and Stabilizers

In certain embodiments, one or more emulsifiers are present in the compositions described herein. In other embodiments, one or more humectants and other stabilizers are also present in the compositions described herein. In some variations, a combination of emulsifiers, humectants and other stabilizers is used.

In some variations, the compositions further comprise a silicone-based emulsifier. Suitable silicone-based emulsifiers may include cetyl PEG/PPG 10/1 Dimethicone, cetyl diglyceryl tris(trimethylsiloxy) silylethyl dimethicone, sorbitan olivate, steareth-20, steareth-2, steareth-21, methyl glucose dioleate, bis-PEG/PPG-14/14 dimethicone (and) dimethicone, dimethicone (and) dimethicone/vinyl dimethicone crosspolymer (and) PEG-10 dimethicone, cyclopentasiloxane (and) dimethicone (and) dimethicone/vinyl dimethicone crosspolymer (and) PEG-10 dimethicone, polyglyceryl-4 isostearate (and) coco-caprylate/caprate (and) hectorite, polyglyceryl-2 dipolyhydroxystearate, lauryl PEG-8 dimethicone, PEG-10 dimethicone, dimethicone (and) caprylyl dimethicone ethoxy glucoside, cylcopentasiloxane (and) caprylyl dimethicone ethoxy glucoside, dimethicone (and) PEG/PPG-18/18 dimethicone, cyclopentasiloxane (and) PEG/PPG-20/15 dimethicone, cyclopentasiloxane (and) PEG-10 dimethicone (and) Bentonite (and) distearyldimonium chloride, dimethicone (and) PEG/PPG-18/18 dimethicone, PEG-8 dimethicone, PEG/PPG-19/19 dimethicone (and) C13-16 isoparaffin (and) C10-13 isoparaffin, mineral oil (and) PEG-15/Lauryl dimethicone crosspolymer, bis-isobutyl PEG/PPG-10/7/dimethicone copolymer, sorbitan olivate, cylcopentasiloxane (and) PEG/PPG-18/18 dimethicone and any combination thereof.

Suitable humectants may include moisturizing humectants (such as glycerin, hydroxyethyl urea, betaine, sodium PCA, sodium-L-Lactate and propanediol), antimicrobial potentiating humectants (such as pentylene glycol, and hexanediol), humectant solvents (such as hexanediol, PEG-4, 8, diprpylene glycol, pentylene glycol, propanediol, butylene glycol, 2-methyl-1,3-propanediol and propylene glycol) and natural based humectants (such as glycerin, pentylene glycol, propanediol, sodium PCA, sodium-L-Lactate and betaine). In other variations, the humectants may include glyceryl esters (such as glycereth-26).

Other suitable stabilizers may include chlorides (such as sodium chloride, potassium chloride, and magnesium chloride), carbonates (such as sodium carbonate, potassium carbonate, magnesium carbonate and propylene carbonate), sulfates (such as sodium sulfate, zinc sulfate, and magnesium sulfate), silica, polymeric thickeners, natural gums, olefin resins, waxes, and any combination thereof.

In some variations, a photostabilizer is used. In certain embodiments, the photostabilizers may also boost SPF.

Skin Feel Enhancers

In certain embodiments, one or more skin feel enhancers are present in the compositions described herein. In some variations, a combination of the skin feel enhancers described herein is used.

Suitable skin feel enhancers may include elastomers and silicones. In some embodiments, the skin feel enhancers are dimethicone fluids. In some variations, the skin feel enhancers are silicon elastomer blends, or polydimethylsiloxane fluids. In certain variations, the skin feel enhancers may be silicone crosspolymers, dimethicone crosspolymers dimethicone/vinyl dimethicone crosspolymers polysilicone-modified silicones, hydrocarbon/silicone crosspolymers, hydrocarbon/dimethicone crosspolymers, hydrocarbon crosspolymers, alkyl/silicone crosspolymers, alkyl/dimethicone crosspolymers, or alkyl crosspolymers. In other variations, the skin feel enhancers are dimethicones, cyclic siloxanes, linear silicones, organofunctional silicones, or organofunctional polydimethylsiloxanes. In other variations, the skin feel enhancers are squalene or hemisqualene. In yet other variations, the skin feel enhancers are isododecane.

In some variations, the skin feel enhancers used in the compositions described herein helps to mitigate product drag, imparts dry cushion during and after rubout, as well as help to anchor the sunscreen film on the skin, and improve product spreadability and dry time.

Excipients

In certain embodiments, the composition further comprises one or more excipients. Suitable excipients may include compounds that can help to improve skin feel, viscosity, stability, SPF boosting capability, and cost. Other suitable excipients may include, for example, esters (such as ethylhexyl isononanoate, C12-15 alkyl benzoate, isopropyl palmitate, diisopropyl adipate, diethylhexyl carbonate), and beeswax.

Vitamins

In certain embodiments, the compositions may further comprise one or more vitamins. For example, in some variations, vitamin E and/or vitamin C may be present in the compositions described herein. In some embodiments, vitamin E and/or vitamin C may be present at about 2%, about 1%, about 0.5%, about 0.25%, about 0.1%, about 0.5%, about 0.25%, or about 0.01% by weight.

Preservation System Components

In certain embodiments, the compositions may further comprise one or more preservation system components. Such preservation system components may include ingredients that provide additional protection against bacterium growth, such as biostatic agents. Suitable preservation system components may include, for example, hydroxyacetophenone and 1,2-hexanediol.

Other Ingredients

In certain embodiments, the compositions may further comprise botanicals, such as plant extracts.

In some variations, the compositions are formulated as lotions or creams, including sunscreen lotions or sunscreen creams. In one variation, the composition is a gel lotion.

In some embodiments, the compositions herein are free of oxybenzone, octinoxate, paraben, phenoxyethanol, colorant, phthalates and formaldehyde donors. In one variation, the composition has less than 0.1, 0.01, or 0.001 wt % of oxybenzone, octinoxate, paraben, phenoxyethanol, colorant, phthalates and formaldehyde donors.

### Composition Properties

The compositions described herein have one or more of the following properties.

Shimmering Effect

In some embodiments, the compositions, when applied on skin, have shimmer and glow effects that provide a product mass tone that disappears on the skin. For example, in one variation, the composition imparts a golden, rosy, coppery, bronzed, or rose gold, shimmering effect on the skin.

Quick Break Effect

In some embodiments, the compositions provided exhibit a quick break effect. For example, in one embodiment, the composition is an oil-in-water quick break emulsion system. In some variations, quick break refers to the instant release of the main part of the water phase in the composition when applied on top of skin.

Physical Stability

In other embodiments, the compositions provided are physically stable. In some variations the compositions exhibit physical stability over a range of temperatures. For example, in certain embodiments, physical stability include is based one or more, or all, of the following conditions:

Condition 1. One month at 50° C. with visual observation;

Condition 2. Three months at 40° C. with visual observation and viscosity check;

Condition 3. Three months at 5° C. with visual observation;

Condition 4. Six months at room temperature with visual observation and viscosity check;

Condition 5. Freeze thaw test with visual observation: 48 hours at −20° C. and 48 hours at room temperature for 4 cycles; and

Condition 6. Transportation test with visual observation: 48 hours at −20° C. and 48 hours at 60° C. for 3 cycles.

Other Properties

In some variations, liquid crystal structures are present in the composition.

In other variations, the sunscreen formulations described herein have a Sun Protection Factor (SPF), measured according to the method prescribed by the US Food and Drug Administration, of at least 15, at least 20, at least 25, at least 30, at least 35, at least 40, at least 45, at least 50, at least 55, or at least 60; or between 15 and 75, or between 15 and 70, between 20 and 65, between 50 and 70, or between 55 and 60. In one variation, the sunscreen formulations described herein have a SPF of about 15, 30 or 50.

In other variations, the sunscreen formulations described herein have a UVA Protection Factor (UVAPF), measured according to the method prescribed by the US Food and Drug Administration, of at least about 16, at least about 17, at least about 18, at least about 19, at least about 20, at least about 21, or at least about 22. In some embodiments, the sunscreen formulations have a UVAPF in a range from about 16 to about 20, or about 16 to about 17. In one variation that may be combined with any of the foregoing variations, the sunscreen formulation has a SPF of 50.

In yet other variations, the UVAPF is higher than one third of the total SPF value, and meets the critical wavelength of higher than 370 nm.

In yet another variation, the sunscreen formulations described herein have a water resistance, measured according to the method prescribed by the US Food and Drug Administration, of up to about 80 minutes, for example, about 40 minutes, about 50 minutes, about 60 minutes, about 70 minutes or about 80 mins. In other variations, the sunscreen formulations described herein have a water resistance of at least 40 minutes. In some embodiments, the sunscreen formulations have a water resistance in a range from about 40 to about 80 minutes, or from about 40 to about 60 minutes.

In another variation, the sunscreen formulations described herein are tear-free.

In yet other variations, the compositions herein have an ultra-light hydrating formula, a powerful water splash which can immediately transform cream to water, and provide an immediate surge of hydration.

### Methods of Manufacturing the Compositions

In certain aspects, provided herein is a method of manufacturing the compositions described herein.

In some embodiments, the method comprises: combining and mixing the oil phase ingredients, while heating to a temperature that is about 5 degrees above the melting point of the highest melting point oil phase ingredient; adding polyacrylate with continuous mixing until well-dispersed; separately combining and mixing the aqueous phase ingredients until uniform; adding pre-dispersion of the special effects pigments into the aqueous phase; adding the hot oil phase to the hot water phase with high shear mixing; removing heat and mixing until uniform, while the batch was cooled; and adding and mixing one or more additional ingredients and water to reach the final weight. In some variations of the foregoing, certain skin feel enhancers may be added as part of the first step involving the combining and mixing of the oil phase ingredients, or post emulsification.

In some variations, the method comprises:

a) combining the organic active agent(s) with other oil-soluble ingredients to form a first mixture, wherein the first mixture comprises an oil phase;

b) mixing and heating the first mixture to a first temperature;

c) adding polyacrylate (such as ammonium acryloyldimethyltaurate/beheneth-25 methacrylate crosspolymer) to the heated first mixture with continuous mixing;

d) combining the water-soluble ingredients to form a second mixture, wherein the second mixture comprises an aqueous phase;

e) mixing and heating the second mixture;

f) applying homogenization if necessary;

g) dispersing the special effects pigments in a solvent to form a separate pre-dispersion

h) mixing and heating the mixture in step (e) to a second temperature to form the hot aqueous phase; adding the pre-dispersion in step (g) with mixing during heating to the mixture;

i) adding the hot oil phase to the hot water phase with high shear mixing; and

j) cooling the mixture in step (i) to a third temperature;

k) post adding ingredients such as preservation system components, fragrances, special effect pigments and skin feel enhancers etc., followed by water and optionally alcohol, to the mixture in step (j) while mixing to produce the composition.

In some variations, the first temperature is between about 180° F. and about 200° F., or between about 185° F. and about 195° F.

In some variations, the second temperature is between about 150° F. and about 180° F.

In some variations, the third temperature is below about 140° F. or below about 104° F.

### Use of the Compositions

In some aspects, provided is a method of applying the compositions described herein. In some embodiments, provided is a method comprising applying any of the compositions described herein onto skin, scalp, and/or hair of a human. In one variation, provided is a method comprising applying any of the compositions onto the face and/or body of a human.

In other aspects, provided is a system to dispense the compositions described herein. In one aspect, provided is an article of manufacture, such as a container comprising any of the compositions described herein, and a label containing instructions for use of the composition. In some embodiments, the container is a tube, containing the composition formulated as a lotion (e.g., tubed lotion).

### ENUMERATED EMBODIMENTS

The following enumerated embodiments are representative of some aspects of the invention.

1. A composition, comprising:

avobenzone; and

a plurality of special effect pigments, wherein at least two of the special effect pigments are (i) a first pigment comprising a layered or deposited metal oxide or salt, or other material, on substrate, and (ii) a second pigment comprising a mineral colorant platelet.

2. The composition of embodiment 1, wherein the composition is free of titanium dioxide.
3. The composition of embodiment 1 or 2, wherein the metal oxide is iron oxide.
4. The composition of any one of embodiments 1 to 3, wherein the second pigment is a pearlescent pigment.
5. The composition of any one of embodiments 1 to 4, wherein the second pigment comprises bismuth oxychloride.
6. The composition of any one of embodiments 1 to 5, the other material in the first pigment comprises silica.
7. The composition of any one of embodiments 1 to 6, wherein the substrate comprises glass, silica, alumina, natural or synthetic mica or synthetic fluorphlogopite or silica, or any combination thereof.
8. The composition of embodiment 7, wherein the substrate comprises mica or synthetic fluorphlogopite or silica, or a combination thereof.
9. The composition of embodiment 7 or 8, wherein the substrate is in the form of flakes, spheres, or platelets.
10. The composition of embodiment 9, wherein the substrate is in the form of platelets.
11. The composition of embodiment 9, wherein the substrate is in the form of flakes.
12. The composition of any one of embodiments 7 to 11, wherein the substrate has a particle size of from 5 μm to 250 μm.
13. The composition of any one of embodiments 7 to 11, wherein the substrate has a particle size of from 5 μm to 20 μm.
14. The composition of any one of embodiments 1 to 13, wherein the composition, when applied to skin, imparts a golden, rosy, coppery, bronzed, or rose gold, shimmering effect on the skin.
15. The composition of any one of embodiments 1 to 14, wherein the composition is free of soluble dyes.
16. The composition of any one of embodiments 1 to 15, further comprising at least one additional sunscreen active agent.
17. The composition of any one of embodiments 1 to 16, wherein the composition is an oil-in-water emulsion.
18. The composition of any one of embodiments 1 to 17, further comprising at least one emulsifier.
19. The composition of any one of embodiments 1 to 17, further comprising a plurality of emulsifiers, wherein at least two of the emulsifiers are (i) a first emulsifier comprising polyacrylate, and (ii) a second emulsifier comprising at least one glucoside and at least one alcohol.
20. The composition of embodiment 19, wherein the composition is an oil-in-water quick break emulsion system.
21. The composition of embodiment 19 or 20, wherein the composition has an oil load of at least 5% based on the total weight of the composition.
22. The composition of embodiment 21, wherein the composition has an oil load of from 5% to 50% based on the total weight of the composition.
23. The composition of any one of embodiments 1 to 22, wherein the composition is physically stable.
24. The composition of any one of embodiments 1 to 23, wherein liquid crystal structures are present in the composition.
25. The composition of any one of embodiments 19 to 24, wherein the first emulsifier comprises a crosspolymer of a sulfonic acid acrylamide monomer or a salt thereof, and an alkyl methacrylate monomer or a salt thereof.
26. The composition of any one of embodiments 19 to 24, wherein the first emulsifier comprises a crosspolymer of an ammonium salt of a sulfonic acid acrylamide monomer, and an alkyl methacrylate monomer.
27. The composition of any one of embodiments 19 to 24, wherein the first emulsifier comprises a crosspolymer of: a monomer of formula (M1) and a monomer of formula (M2), wherein:

the monomer of formula (M1) is:

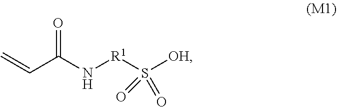

or a salt thereof, wherein R¹ is alkylene;

the monomer of formula (M2) is:

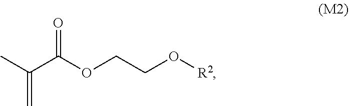

or a salt thereof, wherein R² is alkyl.
   28. The composition of embodiment 27, wherein the monomer of formula (M1) is an ammonium salt.
   29. The composition of embodiment 27 or 28, wherein R¹ is a branched alkylene;
   30. The composition of any one of embodiments 27 to 29, wherein R¹ is —C₁₋₁₀ alkylene.
   31. The composition of any one of embodiments 27 to 30, wherein R² is C₁₅₋₃₀ alkyl.
   32. The composition of any one of embodiments 19 to 31, wherein the second emulsifier comprises at least one glucoside and at least one fatty alcohol.
   33. The composition of embodiment 32, wherein the at least one fatty alcohol are two fatty alcohols.
   34. The composition of embodiment 32 or 33, wherein at least one fatty alcohol is a C₈₊ fatty alcohol.

35. The composition of embodiment 34, wherein the at least one fatty alcohol is a C₁₂₋₂₅ fatty alcohol.

36. The composition of any one of embodiments 32 to 35, wherein at least one glucoside is a glucoside of formula (G1):

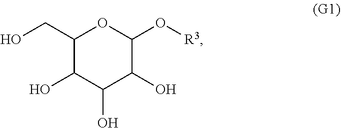

or a salt thereof, wherein R³ is alkyl.
37. The composition of embodiment 36, wherein the glucoside of formula (G1) is a glucoside of formula (G1a):

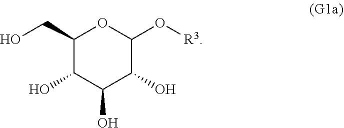

38. The composition of embodiment 36 or 37, wherein R³ is a C₈₊ alkyl.
39. The composition of embodiment 36 or 37, wherein R³ is a C₁₂₋₂₅ alkyl.
40. The composition of any one of embodiments 19 to 24, wherein at least two of the emulsifiers are (i) ammonium acryloyldimethyltaurate/beheneth-25 methacrylate crosspolymer and (ii) arachidyl alcohol, behenyl alcohol and arachidyl glucoside.
41. The composition of any one of embodiments 19 to 24, wherein the first emulsifier comprises:
ammonium acryloyldimethyltaurate/beheneth-25 methacrylate crosspolymer;
acrylates/palmeth-25 acrylate copolymer;
sodium acrylate/acryloyldimethyltaurate/dimethylacrylamide crosspolymer and isohexadecane and polysorbate 60;
ammonium acryloyldimethyltaurate/carboxyethyl acrylate crosspolymer;
acrylate/C10-30 alkyl acrylate crosspolymer;
acrylates copolymer;
carbomer; or
acrylic acid/vinyl pyrrolidone crosspolymer.
   42. The composition of any one of embodiments 19 to 24 and 41, wherein the second emulsifier comprises:
arachidyl alcohol and behenyl alcohol and arachidyl glucoside;
cetearyl glucoside and cetearyl alcohol;
coco-glucoside and coconut alcohol;
myristyl alcohol and myristyl glucoside;
cetearyl alcohol and coco-glucoside;
C14-22 alcohols and C12-20 alkyl glucoside;
cetearyl glucoside and sorbitan olivate and cetearyl alcohol;
cetearyl alcohol and polysorbate 60 and cetearyl glucoside;
hydroxyethyl acrylate/sodium acryloyldimethyl taurate copolymer and cetearyl alcohol and arachidyl alcohol and coco-glucoside and behenyl alcohol and arachidyl glucoside;
cetearyl alcohol and glyceryl stearate and sorbitan stearate and cetearyl glucoside; or
Cannabis sativa seed oil or other oil and C12-18 alkyl glucoside and glyceryl stearate and cetearyl alcohol and stearic acid.
   43. The composition of any one of embodiments 1 to 42, further comprising solvent.
   44. The composition of embodiment 43, wherein the solvent comprises water.
   45. The composition of any one of embodiments 1 to 44, further comprising at least one emollient.
   46. A composition, comprising:

one or more organic active agents, wherein one of the organic active agents is avobenzone;

a plurality of special effect pigments, wherein at least two of the special effect pigments are (i) a first pigment comprising a layered or deposited iron oxide on a substrate, and (ii) a second pigment comprising bismuth oxychloride;

silica; and

a plurality of emulsifiers, wherein at least two of the emulsifiers are (i) a first emulsifier comprising polyacrylate, and (ii) a second emulsifier comprising at least one glucoside and at least one alcohol,

wherein the composition is an oil-in-water emulsion.
47. The composition of embodiment 46, wherein the substrate comprises mica and synthetic fluorphlogopite or silica.
48. The composition of embodiment 46, wherein the substrate comprises mica.
49. The composition of embodiment 46, wherein the substrate comprises synthetic fluorphlogopite and silica.
50. The composition of embodiment 46, wherein the substrate comprises mica and silica.
51. The composition of any one of embodiments 1 to 50, wherein the composition is a sunscreen composition having a SPF rating of at least 15.
52. The composition of any one of embodiments 1 to 51, wherein the composition is a lotion or a cream.
53. The composition of embodiment 50, wherein the composition is a gel lotion.
54. An article of manufacture, comprising:

a container comprising a composition of any one of embodiments 1 to 53; and

a label containing instructions for use of the composition.

55. A method of applying sunscreen, comprising:

applying a composition of any one of embodiments 1 to 53 onto skin, scalp, and/or hair of a human.

### EXAMPLES

The following Examples are merely illustrative and are not meant to limit any aspects of the present disclosure in any way.

### Example 1

### Exemplary Sunscreen Formulation (“Formulation A”)

This example describes an exemplary sunscreen formulation of the disclosure in Table 1 below, and methods of manufacturing thereof.

TABLE 1
Formulation A

| | |
|---|---|
| Ingredient Description | Weight Percent w/w |

Part A Ingredients

| | |
|---|---|
| Avobenzone | 3 |
| Octocrylene | 9 |
| Homosalate | 9 |
| Octisalate | 4.5 |
| Other Oil-Soluble Ingredients (*) | 1-20 |
| Arachidyl Alcohol and Behenyl | 0.1-5 |
| Alcohol and Arachidyl Glucoside | |

Part B Ingredients

| | |
|---|---|
| Ammonium Acryloyldimethyltaurate/Beheneth-25 | 0.1-5 |
| Methacrylate Crosspolymer | |

Part C Ingredients

| | |
|---|---|
| Solvents | 0.1-2 |
| Mica, Iron Oxides, and Silica | 0.01-1 |
| Synthetic Fluorphlogopite, Iron Oxides, Silica | 0.01-1 |

Part D Ingredients

| | |
|---|---|
| Purified Water | 30-70 |
| Other Water-Soluble Ingredient (**) | 0.1-20 |

Part E Ingredients

| | |
|---|---|
| Additional Ingredients post-added (***) | 0.1-15 |
| Bismuth Oxychloride, Mica | 0.01-1 |

Part F Ingredients

| | |
|---|---|
| Purified Water | Q.S. |
| Total | 100.00 |

Other Oil-Soluble Ingredients (*) in Part A may include, for example, anti-oxidants, emollients, film formers, SPF boosters, skin feel enhancers, and other oil-soluble excipients. Other Water-Soluble Ingredient (**) in Part D may include, for example, humectants, thickeners, solvents, SPF boosters, preservatives, and other water-soluble ingredients. Additional Ingredients post-added (***) in Part E may include, for example, solvents, fragrances, skin feel enhancers, and SPF boosters.

### Manufacturing Method

The formulation in Table 1 above was prepared as follows: In a container large enough to hold the oil phase, all the ingredients of Part A (oil phase) were added and mixed while heating the phase to about 5 degrees Fahrenheit above the melting point of the highest melting point ingredient in Part A (e.g., 185° F. to 194° F.). After the oil phase was observed to be within the target temperature range, ammonium acryloyldimethyltaurate/beheneth-25 methacrylate crosspolymer of Part B was added with continuous mixing until well-dispersed. Disperse the special effects pigments in a solvent in Part C to form a separate pre-dispersion. In a container large enough to hold the entire batch, the ingredients of Part D (aqueous phase) were combined with mixing or homogenization until uniform with heating to about the same temperature range as Part A. Add the pre-dispersion in Part C into the aqueous phase in Part D with mixing during heating. The hot oil phase was gradually added to the hot water phase with high shear mixing. Heat was removed, and mixing continued until uniform while the batch was cooled to a temperature ranging from 86° F. to 140° F. The ingredients of Part E were gradually added with mixing, such as alcohol, fragrances, skin feel enhancers etc. Water of Part F was finally added to reach the final weight, and mixed well to form the final composition.

### Quick Break Effect Testing

A sample of Formulation A was applied and spread over a small area on human skin. Instant water release and cooling effect was felt on the skin, indicating that the formulation exhibited a quick break effect.

### Stability Testing

Stability of Formulation A was tested at various temperatures, between −20 and 60° C., including repeated freeze thaw testing and subjecting the sample to long term storage at elevated temperature to justify at least a two year shelf life. The samples were inspected by visual observation and/or rheologically and by other analytical testing. Formulation A was observed to be physically and chemically stable based on the conditions set forth above.

### Example 2

### Exemplary Sunscreen Formulation (“Formulation B”)

This example describes another exemplary sunscreen formulation of the disclosure in Table 2 below, and methods of manufacturing thereof.

TABLE 2
Formulation B

| | |
|---|---|
| Ingredient Description | Weight Percent w/w |

Part A Ingredients

| | |
|---|---|
| Avobenzone | 3 |
| Octocrylene | 9 |
| Homosalate | 9 |
| Octisalate | 4.5 |
| Other Oil-Soluble Ingredients (*) | 1-20 |
| Arachidyl Alcohol and Behenyl | 0.1-5 |
| Alcohol and Arachidyl Glucoside | |

Part B Ingredients

| | |
|---|---|
| Ammonium Acryloyldimethyltaurate/Beheneth-25 | 0.1-5 |
| Methacrylate Crosspolymer | |

Part C Ingredients

| | |
|---|---|
| Solvents | 0.1-2 |
| Mica, Iron Oxides, and Silica | 0.01-1 |

Part D Ingredients

| | |
|---|---|
| Purified Water | 30-70 |
| Other Water-Soluble Ingredient (**) | 0.1-20 |

Part E Ingredients

| | |
|---|---|
| Additional Ingredients post-added (***) | 0.1-15 |
| Bismuth Oxychloride, Mica | 0.01-1 |

Part F Ingredients

| | |
|---|---|
| Purified Water | Q.S. |
| Total | 100.00 |

### Manufacturing Method

The formulation in Table 2 above was prepared according to the method set forth in Example 1 above.

### Quick Break Effect Testing

A sample of Formulation B was applied and spread over a small area on human skin. Instant water release and cooling effect was felt on the skin, indicating that the formulation exhibited a quick break effect.

### Stability Testing

Stability of Formulation B was also tested based on the protocol and criteria set forth in Example 1 above.

Formulation B was observed to be physically stable based on the conditions set forth above.

### Example 3

### Comparative Study of Sensory Attributes of Sunscreen Lotion Formulations

This example compares the sensory attributes of various commercially available sunscreen lotions and Formulation A.

In a consumer panel study, Formulation A was found to possess distinct sensorial attributes distinguishing it from various commercially available sunscreen lotions.

In the study, Formulation A was distinguished as exhibiting better perceived sensorial attributes of wetness, quickbreak, spreadability, absorption, and slipperiness, and less stickiness and stiffness, than various commercially available sunscreen lotions.

Study Design

The study design involved monadic assessments of sunscreen formulations in a randomized and balanced complete block design and estimate mean value for each sensory attribute for each product. A trained consumer panel performed all assessments. Consumer panelists were selected on their ability to detect and discriminate differences in visual and tactile properties. All panelists were validated and extensively trained in evaluation of lotions, sprays, creams, and related product forms.

Attribute intensity was rated on a universal 101 point intensity scale using 1-point increments. Panelists were trained to use the scale in a similar way across panelists and across samples. Use of a universal scale allowed attributes to be compared in intensity to one another, (e.g. comparing intensity of slippery feel to intensity of sticky feel), as well as for comparison of samples within and across studies and products having shared attributes.

All data was collected from the individual respondents, and evaluations were replicated.

### Products Tested

Formulation A was compared with several existing commercial sunscreen lotions. All sunscreen lotions meet an SPF 50 rating.

### Results

Product sensory profiles (individual data means) were used to develop perceptual maps. Perceptual maps of the samples were developed to better understand the relationships among the attributes that define the sensory space of the sample category tested, and where the products fall in that space.

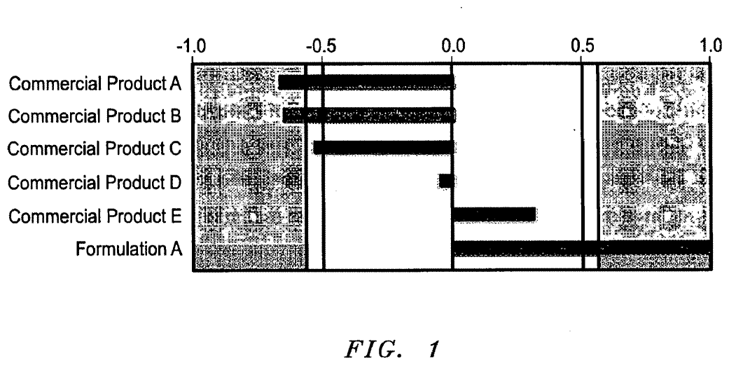

FIG. 1 is a 1-dimensional perceptual map comparing performance during manipulation (Firmness, Stickiness, Peaking, Integrity of Shape & Thickness) with performance during rubout (Wetness, Spreadability, Slipperiness, Oily, Absorbency). Products that fell within the shaded area to the left of the map have the following negative properties: When products were manipulated, they were firmer, stickier, generated higher peaks and maintained their shape. They also felt thicker during rubout. Products that fell within the shaded area to the right of the have the following positive properties: When rubbed into the skin, the product felt more wet, oily and slippery, was easier to spread, but took longer to absorb.

1.-27. (canceled)

28. A sunscreen composition, wherein the composition comprises:
avobenzone; and
a plurality of special effects pigments, at least two of the special effect pigments being (i) a first pigment comprising a layered or deposited metal oxide or salt, or other material, on a substrate, and (ii) a second pigment comprising a mineral colorant platelet.

29. The composition of claim 28, wherein the composition is free of titanium dioxide.

30. The composition of claim 28, wherein the metal oxide is iron oxide.

31. The composition of claim 28, wherein the second pigment is a pearlescent pigment.

32. The composition of claim 28, wherein the second pigment comprises bismuth oxychloride.

33. The composition of claim 28, wherein the other material in the first pigment comprises silica.

34. The composition of claim 28, wherein the substrate comprises one or more of glass, silica, alumina, natural or synthetic mica, synthetic fluorphlogopite.

35. The composition of claim 34, wherein the substrate comprises one or more of mica, synthetic fluorphlogopite, silica.

36. The composition of claim 34, wherein the substrate is in the form of flakes, spheres, or platelets.

37. The composition of claim 34, wherein the substrate has a particle size of from 5 μm to 250 μm.

38. The composition of claim 28, wherein the composition is free of soluble dyes.

39. The composition of claim 28, wherein the composition further comprises at least one additional sunscreen active agent.

40. The composition of claim 28, wherein the composition is an oil-in-water emulsion.

41. The composition of claim 28, wherein the composition further comprises a plurality of emulsifiers, at least two of the emulsifiers being (i) a first emulsifier comprising polyacrylate, and (ii) a second emulsifier comprising at least one glucoside and at least one alcohol.

42. A sunscreen composition, wherein the composition is an oil-in-water emulsion and comprises:
one or more organic active agents, one of the organic active agents being avobenzone;
a plurality of special effects pigments, at least two of the special effect pigments being (i) a first pigment comprising a layered or deposited iron oxide on a substrate, and (ii) a second pigment comprising bismuth oxychloride;
silica; and
a plurality of emulsifiers, at least two of the emulsifiers being (i) a first emulsifier comprising polyacrylate, and (ii) a second emulsifier comprising at least one glucoside and at least one alcohol.

43. The composition of claim 42, wherein the substrate comprises mica.

44. The composition of claim 42, wherein the composition is a lotion or a cream.

45. The composition of claim 28, wherein the composition is a gel lotion.

46. An article of manufacture, comprising:
a container comprising the composition of claim 28; and
a label containing instructions for use of the composition as sunscreen composition.

47. A method of protecting human skin, scalp and/or hair from sun radiation, wherein the method comprises applying the composition of claim 28 onto skin, scalp, and/or hair of a human.
